(19) United States
(12) Patent Application Publication
(10) Pub. No.: US 2026/0283642 A1
(43) Pub. Date: Sep. 24, 2026

(54) NEEDLE GUIDING SYSTEM, A NEEDLE GUIDE AND A METHOD FOR USING THE NEEDLE GUIDING SYSTEM IN GUIDING A NEEDLE FOR NEEDLE INSERTION OF A PATIENT

(71) Applicants: Singapore Health Services Pte Ltd, Singapore (SG); HiCura Medical Pte. Ltd., Singapore (SG)

(72) Inventors: Hon Sen TAN, Singapore (SG); Ban Leong SNG, Singapore (SG); Tiong Heng Alex SIA, Singapore (SG); Cailin NG, Singapore (SG)

(21) Appl. No.: 18/998,007

(22) PCT Filed: Aug. 2, 2023
(86) PCT No.: PCT/SG2023/050535

§ 371 Date: Jan. 23, 2025

(30) Foreign Application Priority Data
Aug. 4, 2022 (SG) 10202250668N

(51) Int. Cl. A61B 17/34; A61B 90/00

(52) U.S. Cl. CPC A61B 17/3403; A61B 2017/3407; A61B 2017/3413; A61B 2090/067; A61B 2090/395

(57) ABSTRACT

A needle guiding system is described. The needle guiding system comprises a marking device 402, the marking device 402 comprising: a base 602; an angle scale 604 having angle markings indicating angle positions of an imaging probe with respect to a plane of the base 602; and an angle indicator 606 adapted to indicate a probe angle on the angle scale 604, wherein the angle indicator 606 is configured to move in tandem with the imaging probe to identify an insertion angle for needle insertion when the imaging probe is aligned at the insertion angle at an insertion site. A needle guide 404 and a method for using the needle guiding system in guiding a needle for needle insertion of a patient are also described.

### TECHNICAL FIELD

The present disclosure relates to a needle guiding system, a needle guide and a method for using the needle guiding system in guiding a needle for needle insertion of a patient.

### BACKGROUND

Lumbar neuraxial procedures are commonly performed for therapeutic and diagnostic indications, including surgical anaesthesia, postoperative and labour analgesia, chronic pain management, and lumbar punctures. In particular, neuraxial anaesthesia is the standard technique for Caesarean delivery worldwide, with more than 1.4 million procedures performed annually in the USA alone. Furthermore, neuraxial analgesia is considered the gold standard for labour analgesia, and is utilized by over 60% of labouring women in the USA. Locally in Singapore, at KK Women's and Children's Hospital, approximately 8,000 neuraxial procedures are performed annually for labour analgesia or Caesarean delivery. Notably, the cost of epidural complications, including the time lost due to failed procedures, exceeds $1.5 billion annually in the USA alone. Hence, improving the safety, effectiveness, and reliability of lumbar neuraxial procedures will benefit a large patient population and reduce healthcare costs.

During the neuraxial procedure, it is crucial to accurately determine an optimal needle insertion point and angulation, in order to minimize procedural time and the risk of complications (such as multiple needle punctures or re-direction, procedural pain, bleeding, and neurological injury). At present, neuraxial procedures are typically performed “blind”, via palpation and identification of anatomical landmarks. However, multiple studies have shown that landmark palpation is highly inaccurate, especially in patients with obesity, tissue edema, or abnormal spine anatomy.

The recent development of neuraxial ultrasonography aids identification of anatomical landmarks and improves an accuracy and angulation of needle insertion during the neuraxial procedure. Compared to landmark palpation, substantial evidence has demonstrated that neuraxial ultrasonography reduces the number of puncture attempts, shortens procedural time, and decreases incidences of complications such as post-dural headache and vascular puncture. Further, ultrasonography is readily available in most tertiary healthcare settings, and does not involve exposure to harmful radiation. This makes it suitable for routine clinical use in obstetric and paediatric settings. Accordingly, this technique is advocated by national guidelines and is increasingly adopted by anesthesiologists to aid needle placement during neuraxial procedures. Despite these advantages, neuraxial ultrasonography in clinical practice is limited by difficulties. For example, it is difficult to translate information derived from the neuraxial ultrasonography into accurate and precise measurements for a needle insertion site and to demarcate the needle insertion point/site precisely. Further, it is also difficult to maintain the needle at an optimal angulation during the neuraxial procedure.

It is therefore desirable to provide a needle guiding system, a needle guide and a method for using the needle guiding system in guiding a needle for needle insertion of a patient which addresses the aforementioned problems and/or provides a useful alternative.

Furthermore, other desirable features and characteristics will become apparent from the subsequent detailed description and the appended claims, taken in conjunction with the accompanying drawings and this background of the disclosure.

### SUMMARY

Aspects of the present application relate to a needle guiding system, a needle guide and a method for using the needle guiding system in guiding a needle for needle insertion of a patient.

In accordance with a first aspect, there is provided a needle guiding system comprising a marking device, the marking device comprises: a base; an angle scale having angle markings indicating angle positions of an imaging probe with respect to a plane of the base; and an angle indicator adapted to indicate a probe angle on the angle scale, wherein the angle indicator is configured to move in tandem with the imaging probe to identify an insertion angle for needle insertion when the imaging probe is aligned at the insertion angle at an insertion site.

Thus, the described embodiment provides a needle guiding system comprising the marking device. The needle guiding system may be used for demarcating an insertion site or position, as well as an insertion angle for needle insertion. By having the angle indicator configured to move in tandem with the imaging probe and adapted to indicate a probe angle on the angle scale, the insertion angle for needle insertion can be identified or measured when the imaging probe is aligned at the insertion angle at the insertion site. The identified insertion angle can then be subsequently used for performing the needle insertion. This improves an accuracy of the identified insertion angle for needle insertion, thereby minimising complications related to inaccurate needle insertion. Further, the angle indicator is adapted to move in tandem with the imaging probe and is configured to indicate a probe angle on the angle scale of the marking device. This eliminates additional steps for measuring and marking the insertion angle by other means, because as soon as the imaging probe is aligned at the insertion site and at the insertion angle, the angle indicator will identify the insertion angle for needle insertion on the angle scale.

The system may comprise a probe interface adapted to operationally connect the imaging probe to the angle indicator. The probe interface may be adapted to be detachable from the angle scale. The probe interface may include a probe holder configured to hold the imaging probe and may comprise the angle indicator. The probe interface may be adapted to be customisable to fit imaging probes of various shapes and sizes.

The angle scale may be adapted to be movable with respect to the base so that an axis passing through a middle of the imaging probe is aligned to the insertion site for different widths of the imaging probe.

The base may comprise width markings corresponding to respective positions of the movable angle scale for indicating a width of the imaging probe. The base may comprise at least one protrusion for generating at least one mark on a skin of the patient to indicate a position of the base with respect to the skin of the patient when the imaging probe is aligned at the insertion angle at the insertion site.

The system may comprise a needle guide, wherein the needle guide may comprise: a guiding angle scale having guide angle markings corresponding to the angle markings of the marking device for indicating the angle positions of the imaging probe; and an insertion slot for aligning a needle to the insertion site. The needle guide may comprise needle slots at each of the guide angle markings for aligning the needle to a corresponding angle position. The needle guide may comprise at least one opening corresponding to the at least one protrusion of the base for aligning the needle guide to the insertion site using the at least one mark on the skin of the patient.

The guiding angle scale may be adapted to hinge at a needle guide base so that the guiding angle scale is collapsible to be in plane with the needle guide base. The guiding angle scale may be adapted to be detachable from a needle guide base. The guiding angle scale may be in the form of an arch and may be adapted to allow the insertion site to be visible from both sides of the arch.

The angle scale may be adapted to face a user of the marking device. The angle positions may be regularly spaced at an angular displacement of 5 degrees. The angle indicator may be adapted to be aligned along a longitudinal mid-plane of a face of the imaging probe.

The imaging device may include an ultrasound probe.

In accordance with a second aspect, there is provided a needle guide comprising: a guiding angle scale having guide angle markings adapted to indicate angle positions of an imaging probe for identifying an insertion angle for needle insertion; and an insertion slot for aligning a needle to an insertion site for the needle insertion.

The needle guide may comprise needle slots at each of the guide angle markings for aligning the needle to a corresponding angle position.

The guiding angle scale may be adapted to hinge at a needle guide base so that the guiding angle scale is collapsible to be in plane with the needle guide base. This advantageously allow the guiding angle scale to be flat-packed. Further, the guiding angle scale may be collapsed if required for performing local anaesthesia so that the guiding angle scale will not be obstructing the local anaesthesia process.

The guiding angle scale may be detachable from a needle guide base. The guiding angle scale may be in the form of an arch and is adapted to allow the insertion site to be visible from both sides of the arch.

In accordance with a third aspect, there is provided a method of using a needle guiding system for guiding a needle for needle insertion of a patient, the needle guiding system comprising a marking device and a needle guide, wherein the marking device comprises a base, an angle scale having angle markings indicating angle positions of an imaging probe with respect to a plane of the base and an angle indicator adapted to indicate a probe angle on the angle scale, wherein the angle indicator is configured to move in tandem with the imaging probe, and wherein the needle guide comprises a guiding angle scale having guide angle markings corresponding to the angle markings of the marking device for indicating the angle positions of the imaging probe and an insertion slot for aligning a needle to an insertion site, wherein the base of the marking device comprises at least one protrusion and the needle guide further comprises at least one opening corresponding to the at least one protrusion of the base, the method comprising: aligning the imaging probe to the insertion site and at an insertion angle; using the angle indicator of the marking device to record the insertion angle associated with the angle markings of the angle scale; generating at least one mark on a skin of the patient using the at least one protrusion of the base of the marking device for marking the insertion site with respect to the base of the marking device; replacing the marking device with the needle guide; aligning the needle guide to the insertion site using the at least one opening of the needle guide and the at least one mark on the skin of the patient; and placing the needle in the insertion slot of the needle guide and at a guide angle marking corresponding to the insertion angle to guide the needle for needle insertion of the patient.

Embodiments therefore provide a needle guiding system, a needle guide and a method for using the needle guiding system in guiding a needle for needle insertion of a patient. Particularly, the needle guiding system can be used for demarcating an insertion site or position, as well as an insertion angle for needle insertion in a neuraxial procedure. By having the angle indicator of the marking device configured to move in tandem with the imaging probe and adapted to indicate a probe angle on the angle scale, the insertion angle for needle insertion can be identified or measured when the imaging probe is aligned at the insertion angle at the insertion site. This eliminates additional steps for measuring and marking the insertion angle by other means, because as soon as the imaging probe is aligned at the insertion site and at the insertion angle, the angle indicator will identify the insertion angle for needle insertion on the angle scale. In embodiments where a needle guide is provided, the needle guide includes guide angle markings adapted to indicate angle positions of the imaging probe for identifying the insertion angle for needle insertion and the insertion slot for aligning the needle to the insertion site. The needle guide advantageously aids to translate the measured insertion angle identified using the marking device into actual insertion angle identified on the guide angle markings for guiding the needle during needle insertion. The needle guide also helps in maintaining or guiding the needle at the same angle during the needle insertion process. Further, the insertion slot of the needle guide also aids in marking the insertion site/point for needle insertion as identified by the imaging probe. This improves an accuracy of the identified insertion angle for needle insertion, thereby minimising complications related to inaccurate needle insertion. When the marking device and the needle guide work in tandem, the base of the marking device may comprise at least one protrusion and the needle guide may comprise at least one opening corresponding to the at least one protrusion of the base. In this way, the at least one protrusion of the base of the marking device can be used to mark the insertion site on a skin of the patient with respect to the base of the marking device, and the needle guide can then be aligned to the insertion site using the at least one opening of the needle guide and the at least one mark on the skin of the patient. The needle guide can therefore be placed accurately on the area for insertion, having the insertion slot aligned to the insertion site/point. Further, by having the marking device and the needle guide as individual components of the needle guiding system, the marking device can be adapted to be used repeatably as a non-sterile component while the needle guide can be adapted to be a one-use sterile item. This aids in ensuring that the entire neuraxial procedure can be performed in an efficient manner while maintaining hygiene for the needle insertion step. The marking device can also be used repeatedly which means less wastage and less cost. The needle guiding system may include a probe interface adapted to be customisable to fit imaging probes of different sizes and/or shapes. This improves a variability of the needle guiding system.

### BRIEF DESCRIPTION OF THE DRAWINGS

Embodiments will now be described, by way of example only, with reference to the following drawings, in which:

FIGS. 1A, 1B, 1C, 1D, 1E and 1F show diagrams illustrating a typical clinical workflow of neuraxial ultrasonography for needle insertion, where FIG. 1A shows a diagram illustrating a setup of an ultrasound system, FIG. 1B shows a diagram illustrating a patient being prepared for needle insertion, FIG. 1C shows a diagram illustrating a use of the ultrasound system for locating a needle insertion site, FIG. 1D shows a diagram illustrating marking of the needle insertion site, FIG. 1E shows a diagram illustrating disinfection of the needle insertion site and FIG. 1F shows a diagram illustrating insertion of a needle at the needle insertion site;

FIGS. 2A, 2B and 2C show illustrations of a conventional method for determining a needle insertion site with neuraxial ultrasonography, where FIG. 2A shows an illustration of marking during a vertical scan, FIG. 2B shows an illustration of marking during a horizontal scan, and FIG. 2C shows an illustration of different insertion sites being intersections of vertical markings and horizontal markings;

FIGS. 3A and 3B show illustrations of a problem faced using the conventional method for marking the needle insertion site of FIG. 2C, where FIG. 3A shows an illustration of the markings on a skin of the patient and FIG. 3B shows an illustration of the markings of FIG. 3A being erased during skin sterilization;

FIG. 4 shows a photograph of the needle guiding system of FIGS. 4A and 4B in accordance with a first embodiment;

FIGS. 5A, 5B and 5C show diagrams of a needle guiding system in accordance with the first embodiment, where FIG. 5A shows a diagram of a marking device of the needle guiding system, FIG. 5B shows a diagram of a needle guide of the needle guiding system, and FIG. 5C shows a diagram of a connector of the needle guiding system for connecting an imaging probe (e.g. an ultrasound probe) to the marking device;

FIGS. 6A and 6B show diagrams of the marking device of FIG. 5A and the connector of FIG. 5C, respectively, in accordance with the first embodiment;

FIGS. 7A and 7B show diagrams of the needle guide of FIG. 5B in accordance with the first embodiment, where FIG. 7A shows a diagram of the needle guide in a ready to use state and FIG. 7B shows a diagram of the needle guide as formed or moulded;

FIGS. 8A, 8B and 8C show illustrations of a method for marking a needle insertion site and an insertion angle using the needle guiding system of FIGS. 5A, 5B and 5C in accordance with an embodiment, where FIG. 8A shows an illustration of determining the needle insertion site and the insertion angle using an imaging probe, FIG. 8B shows an illustration of marking the needle insertion site and recording the insertion angle using the marking device of FIG. 5A, and FIG. 8C shows an illustration of guiding a needle for needle insertion at the needle insertion site and at the insertion angle using the needle guide of FIG. 5B;

FIG. 9 is a flow chart showing steps of a method for determining a needle insertion site using neuraxial ultrasonography and marking the needle insertion site using e.g. the needle guiding system of FIGS. 5A, 5B and 5C in accordance with an embodiment (do note that this method is also applicable to other embodiments of the needle guiding system as described below);

FIGS. 10A, 10B, 10C, 10D and 10E show diagrams illustrating a workflow for marking of a needle insertion site and an insertion angle using the needle guiding system of FIGS. 5A, 5B and 5C in accordance with an embodiment, where FIG. 10A shows a diagram illustrating alignment of the marking device at locating the insertion site and recording of the insertion angle using neuraxial ultrasonography, FIG. 10B shows a diagram illustrating marking of a patient by pressing the marking device of FIG. 5A against a skin of the patient at the insertion site, FIG. 10C shows a diagram illustrating marks left on the skin of the patient using the marking device, FIG. 10D shows a diagram illustrating alignment of the needle guide of FIG. 5B at the insertion site using the marks left on the skin of the patient, and FIG. 10E shows a diagram illustrating insertion of a needle at the insertion site using the needle guide;

FIGS. 11A and 11B show illustrations of a usability study performed using the needle guiding system of FIGS. 5A, 5B and 5C in accordance with an embodiment, where FIG. 11A shows an illustration of aligning the marking device of FIG. 5A to an insertion site and recording an insertion angle using an imaging probe, and FIG. 11B shows an illustration of a mock needle insertion using the needle guide of FIG. 5B;

FIGS. 12A and 12B shows illustrations of a usability study performed using the conventional method for determining of the needle insertion site with neuraxial ultrasonography and marking of the needle insertion site using a marker pen, where FIG. 12A shows an illustration of marking an insertion site using the marker pen and FIG. 12B shows an illustration of a mock needle insertion by estimating an insertion angle;

FIGS. 13A, 13B and 13C show diagrams of a marking device of a needle guiding system in accordance with a second embodiment, where FIG. 13A shows a diagram of the marking device with a modified angle scale and a detachable probe interface in an assembled state, FIG. 13B shows a diagram of the marking device with the modified angle scale and the detachable probe interface in a disassembled state, and FIG. 13C shows a diagram of the marking device with an imaging probe attached;

FIGS. 14A and 14B show diagrams of a needle guide of the needle guiding system in accordance with the second embodiment, where FIG. 14A shows a diagram of the needle guide in a folded state, and FIG. 14B shows a diagram of the needle guide in a ready-to-use state with a needle placed in a needle slot at a corresponding angle and through an insertion slot;

FIGS. 15A, 15B and 15C show diagrams of a marking device of a needle guiding system in accordance with a third embodiment, where FIG. 15A shows a diagram of a perspective view of the marking device with a modified angle scale, FIG. 15B shows a diagram of a bottom view of the marking device to illustrate an adjustability of the angle scale for imaging probes of different widths, and FIG. 15C shows a diagram of the marking device aligned beside an imaging probe for direct reading of an insertion angle;

FIGS. 16A and 16B show diagrams of a needle guide of the needle guiding system in accordance with the third embodiment to illustrate that a needle for needle insertion is visible at both sides of an arch of the needle guide;

FIGS. 17A and 17B show diagrams of a marking device of a needle guiding system in accordance with a fourth embodiment, where FIG. 17A shows a diagram of a perspective view of the marking device with a further modified angle scale and a detachable probe interface in an assembled state, and FIG. 17B shows a diagram of the marking device with an imaging probe attached using the detachable probe interface;

FIGS. 18A and 18B shows diagrams of the detachable probe interface of the marking device of FIG. 17A in accordance with the fourth embodiment, where FIG. 18A shows a diagram illustrating a probe side of the detachable probe interface for attaching an imaging probe and FIG. 18B shows a diagram illustrating a marking device side of the detachable probe interface for attaching the probe holder to the marking device;

FIGS. 19A and 19B show diagrams of the needle guiding system in accordance with the fourth embodiment, where FIG. 19A shows a diagram of the marking device of the needle guiding system and FIG. 19B shows a diagram of a needle guide of the needle guiding system;

FIGS. 20A and 20B shows diagrams of two different embodiments of the marking device (i.e. the first embodiment and the fourth embodiment) for comparison; and

FIGS. 21A and 21B show photographs of a needle guide in accordance with a fourth embodiment.

### DETAILED DESCRIPTION

Exemplary embodiments relating to a needle guiding system, a needle guide and a method for using the needle guiding system in guiding a needle for needle insertion of a patient are described.

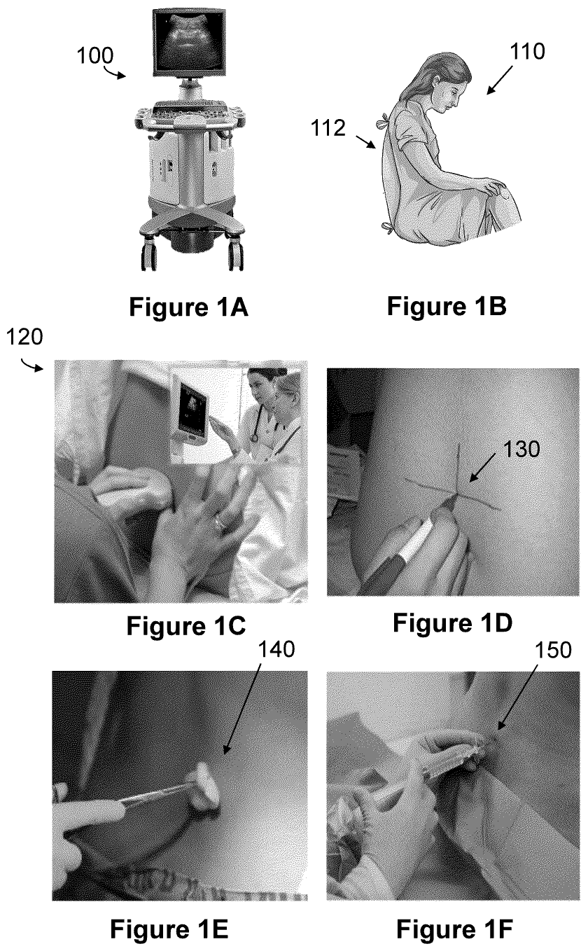

FIGS. 1A to 1F, 2A to 2C and 3A to 3B relate to current practices for neuraxial ultrasonography and have been included to illustrate gaps and shortcomings in relation to present state of the art.

### Gaps and Shortcomings of Current Practice

Notwithstanding its advantages, neuraxial ultrasonography in clinical practice is limited by difficulties with 1) precise demarcation of the needle insertion point (position marking) and 2) accurate measurement and maintenance of optimal needle angulation during the needle insertion (angular measurement).

FIGS. 1A, 1B, 1C, 1D, 1E and 1F show diagrams illustrating a typical clinical workflow of neuraxial ultrasonography for needle insertion at an interspinous space (e.g. a L3/L4 interspinous space).

Firstly, an ultrasound machine 100 is setup beside a patient 110 as shown in FIGS. 1A and 1B. The patient 110 is seated with his/her back 112 hunched in preparation for needle insertion. This is shown in FIG. 1B. FIG. 1C shows a diagram 120 illustrating use of an ultrasound system for locating a needle insertion site. At this stage, a clinician applies ultrasound gel and scans the patient's lumbar area to look out for several spinal landmarks that will identify an optimal needle insertion spot and an insertion angle. Typically for epidural neuraxial insertion, the landmarks are the L3/L4 interspinous space, a midline of the spine and an epidural space. Once the optimal needle insertion spot has been located using the ultrasound, the clinician uses a skin marker 130 to mark the centre or middle of the probe on the patient's back for marking the needle insertion site. This is shown in FIG. 1D. The needle insertion site is then disinfected by wiping skin 140 with antiseptic solutions as shown in FIG. 1E, before inserting the needle at the needle insertion site 150 as shown in FIG. 1F.

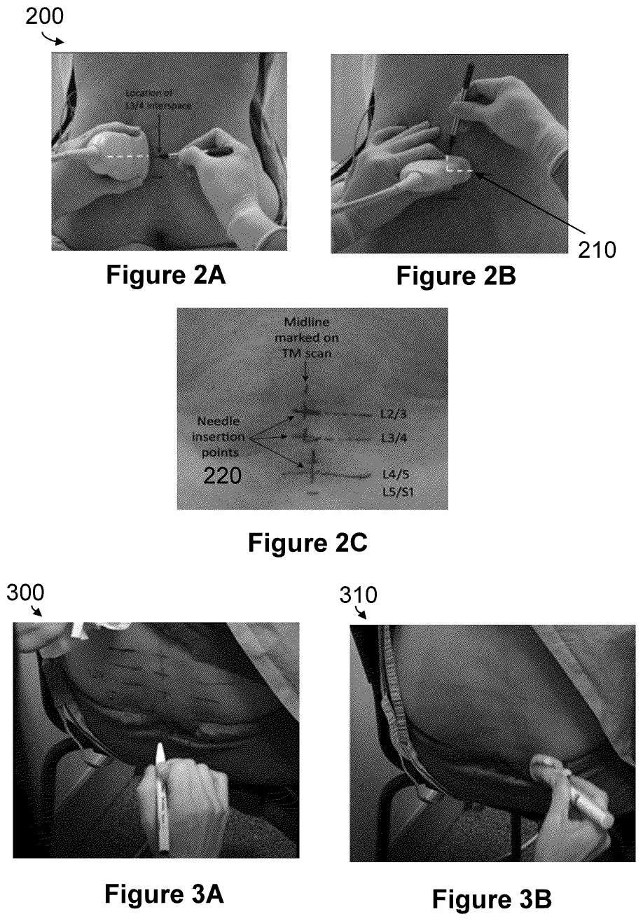

FIGS. 2A, 2B and 2C show illustrations of a conventional method for determining the needle insertion site with neuraxial ultrasonography.

An optimal needle insertion point is conventionally determined via two separate ultrasonographic scans. FIG. 2A shows an illustration 200 of marking during a first vertical scan. The aim of the first vertical scan is to identify appropriate points of needle insertion on the vertical axis (i.e. a longitudinal axis along a length of a spine of the patient). With the patient in a sitting position, an ultrasound probe is placed vertically over the sacrum, and moved cephalad to identify consecutive spaces between the vertebrae (interspaces) until the interspace chosen for needle placement is centred in the middle of the screen. This level on the vertical axis is denoted by marking the patient's skin corresponding to a middle along a transverse plane of the ultrasound probe, as illustrated in FIG. 2A. Next, a second scan is performed to identify the spinal midline by rotating the ultrasound probe 90 degrees to scan in the horizontal axis 210 of the spine (i.e. a transverse axis perpendicular to the length of the spine) and moving it horizontally to align the spinal midline to the middle of the screen. This position on the horizontal axis is then denoted by marking the patient's skin corresponding to the middle of the ultrasound probe, as illustrated in FIG. 2B. Finally, the needle insertion point is determined by intersecting the vertical and horizontal markings, as shown in FIG. 2C. FIG. 2C also shows different needle insertion sites 220 as marked out, for example, interspinous spaces between L2/L3, L3/L4 and L4/L5.

There are issues and problems, however, faced by clinicians with marking the needle insertion sites as illustrated using the above method. For example, marking the patient's skin with surgical marker is difficult over areas smeared with ultrasound gel (required to obtain good images during ultrasonography). Further, the markings made (e.g. as shown in FIG. 2C) may be erased or wiped away during skin sterilization. This is illustrated using FIGS. 3A and 3B, where FIG. 3A shows an illustration 300 of the markings of needle insertion sites on the skin of the patient and FIG. 3B shows an illustration 310 of the markings of FIG. 3A being erased when the skin around the needle insertion sites is disinfected. This results in inaccurate skin markings and an increase in procedure time. In addition, the midpoints of most available ultrasound probes are not indicated on the probe itself. Consequently, the clinician is required to estimate the middle of the ultrasound probe when making the skin markings, resulting in imprecision between the resultant markings and the actual midpoint of the ultrasound probe.

Another limitation pertains to accurately determining and maintaining optimal needle angulation during the procedure. During ultrasonography, the clinician optimizes the view of the selected interspace by tilting the probe, and the angle of the ultrasound probe relative to the patient's skin determines the required needle angulation during insertion. Due to the inability to precisely measure the angle of the probe, the clinician can only visually estimate the probe angle and reproduce the estimated angulation during needle insertion. The difficulty and imprecision of reproducing the correct angulation is exacerbated by the interceding steps of the neuraxial procedure, including skin sterilization and preparing the drugs and equipment needed, after which the clinician may have forgotten the required needle angulation as identified using the ultrasound probe.

There are various ways for marking a needle insertion site or measuring an insertion angle for needle insertion, but each has their own issues.

For example, one practice for position marking involves using the clinician's fingertips to denote the horizontal and vertical axes to determine the needle insertion point. This may involve encircling the probe by the clinician's hand with the index finger, and the thumb centred on the middle of the ultrasound probe. After both the vertical and horizontal scans (e.g. as shown in relation to FIGS. 2A and 2B) are performed, the probe is removed while the fingertips remain on the skin (i.e. the fingers are in the positions as shown in FIG. 2B with the probe removed). A horizontal line between the fingertips denotes the horizontal axis of the spine, and the estimated midpoint between the fingertips denotes the vertical axis of the spine, which will then be marked as the needle insertion point. Although this approach may reduce the time required for skin marking, it potentially increases the inaccuracy of needle insertion due to fingertip thickness, movement of the fingertips, and inaccurate visual estimation of the midline between the fingertips.

Another practice for measuring an insertion angle for needle insertion is by using a smartphone equipped with accelerometers. However, the smartphone in this case measures the probe angle relative to gravity rather than to the patient's skin and may lead to inaccuracies if the patient changes his/her posture after the angle measurement. Furthermore, use of the smartphone requires assistance from an additional clinician, complicating the marking process and increasing the procedure time. Having the additional clinician for the procedure also makes it impractical, thereby limiting its widespread adoption in clinical practice.

Although solutions such as the above have been proposed, they are also associated with significant limitations and do not address the limitations with neuraxial ultrasonography, including difficulties with precise demarcation of needle position and maintaining optimal needle angulation during the procedure.

In view of the above, it is therefore desirable to provide a system or device which can accurately demarcate the needle insertion point, measure the needle insertion angle required, and is compatible with a range of ultrasound probes or other imaging probes.

Exemplary embodiments as described below relate to a needle guiding system, a needle guide and a method for using the needle guiding system in guiding a needle for needle insertion of a patient for addressing the aforementioned problems and/or provides a useful alternative.

### Concept Design and First Needle Guiding System Prototype

To develop a first needle guiding system prototype (or Position and Angle Marking System (PAMS)), comprehensive user feedback with anesthesiologists who routinely perform neuraxial procedures was received to provide a list of ideal parameters or requirements for PAMS:
Able to locate a centre of the probe in both vertical and horizontal directions;
Able to leave a skin marking that will remain visible for >5 mins and up to 15 mins;
Able to measure and record an angle of the probe in relation to a back of the patient or a skin of the insertion site;
Able to place a needle at a correct position and angle, with ±5 mm position marking accuracy and ±5° angular accuracy; and
Compatible with ultrasound probes of varying sizes and shapes.

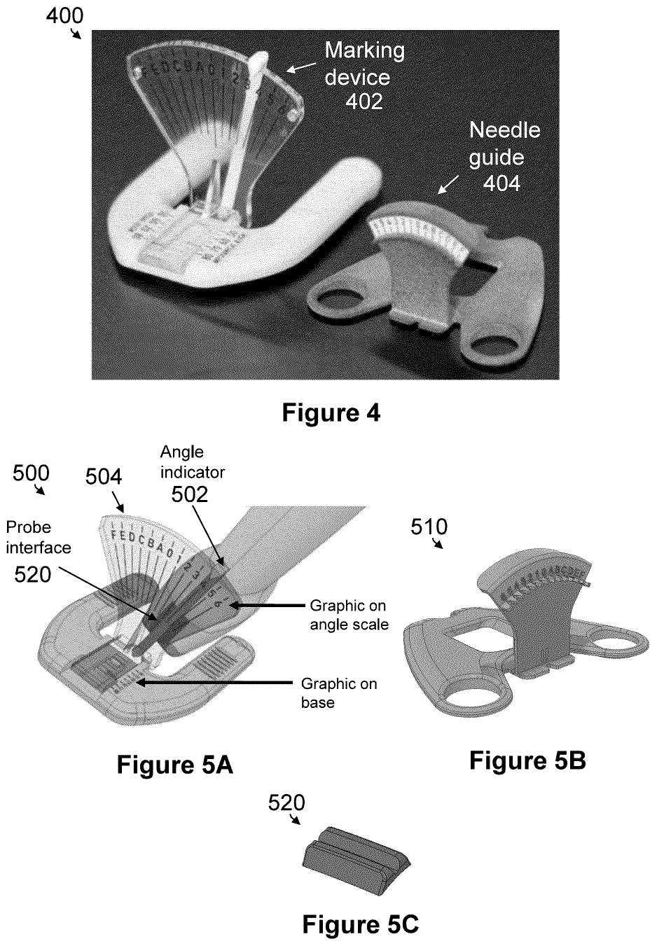

Based on these requirements, the following first prototype was designed and fabricated. The first embodiment of the needle guiding system comprises mainly two parts, a marking device and a needle guide. FIG. 4 shows a photograph 400 of the needle guiding system in accordance with the first embodiment, comprising the marking device 402 and the needle guide 404. The marking device 402 functions to mark an optimal needle insertion site/point and to record an insertion angle (or angulation) for the needle insertion, while the needle guide 404 functions to maintain a needle position and insertion angle during the needle insertion or neuraxial procedure.

FIGS. 5A, 5B and 5C show diagrams of the needle guiding system in accordance with this first embodiment, where FIG. 5A shows a diagram of a marking device 500, FIG. 5B shows a diagram of a needle guide 510, and FIG. 5C shows a diagram of a probe interface 520 (in the form of a connector) of the needle guiding system for connecting an imaging probe (e.g. an ultrasound probe) to the marking device 500. The connecting of the imaging probe to the marking device 500 using the connector is also shown in FIG. 5A. Also shown in relation to FIG. 5A is an angle indicator 502 adapted to indicate or identify an optimal insertion angle on the angle scale 504 for needle insertion. In an embodiment, graphics on the angle scale 504 and a base of the marking device can be screen or tampo printed.

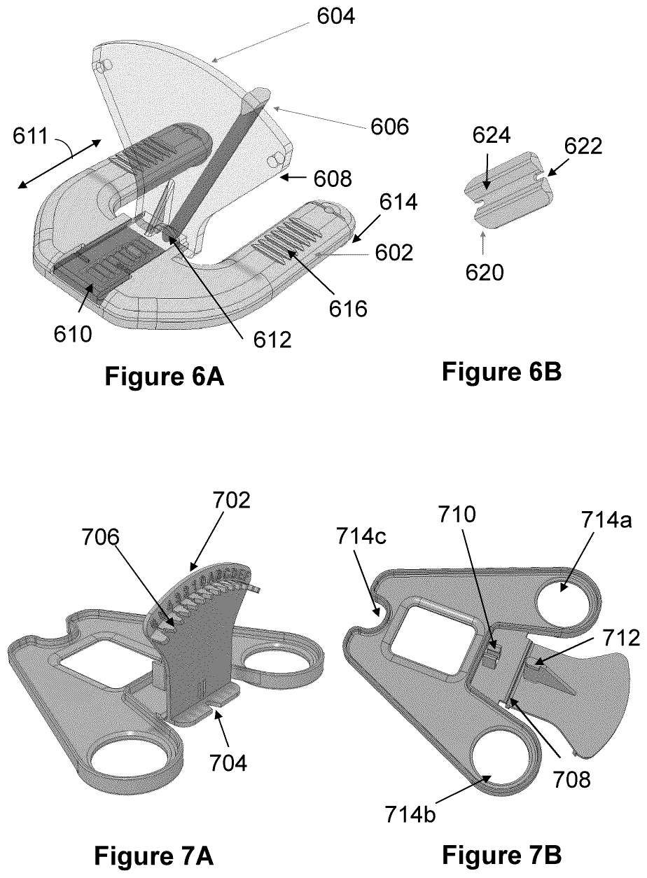

Details of the parts of the needle guiding system as well as the material of choice and their finishes in accordance with the first embodiment are discussed in relation to FIGS. 6A-6B and 7A-7B.

FIGS. 6A and 6B show diagrams of the marking device 500 of FIG. 5A and the probe interface 520 of FIG. 5C, respectively, in accordance with the first embodiment.

As shown in FIG. 6A, the marking device 500 includes a base 602, an angle scale 604 and an angle indicator 606. The base 602 in the present embodiment is in a form of a C-shape. The angle scale 604 is detachable and slidable with respect to the base 602, and is operationally connected to the base 602 at a middle of the bend of the C-shaped base 602. The angle scale 604 in the present embodiment has a fan-shaped indicator plane 608 having angle markings (see e.g. FIG. 5A) indicating angle positions of an imaging probe with respect to a plane of the base 602. The angle positions in the present embodiment are regularly spaced at an angular displacement of 5 degrees. In the present embodiment, the fan-shaped indicator plane 608 is perpendicular to a plane of the base 602. The angle scale also has an angle scale base 610 functions to connect the angle scale 604 to the base 602. The angle scale base 610 is adapted to be slidable with respect to the base 602 so that when an imaging probe is connected to the marking device, a middle of the imaging probe is always aligned to a specific point (the insertion site) with respect to the base 602 for different widths of the imaging probe. For this, the base 602 comprises width markings corresponding to respective positions of the movable angle scale 604 for indicating a width of the imaging probe. In other words, the angle scale 604 together with its angle scale base 610 are movable/slidable along a longitudinal axis 611 of the base 602 for matching a width of the imaging probe. In the present embodiment, the angle scale 604 together with its angle scale base 610 are movable/slidable at interval steps of 5 mm, although it should be appreciated that other interval steps (e.g. 1 mm, 2 mm, 3 mm or 10 mm etc. may be used). In this manner, the marking device can be compatible (or used with) imaging probes of different widths/sizes. This is more clearly shown in FIGS. 4 and 5A.

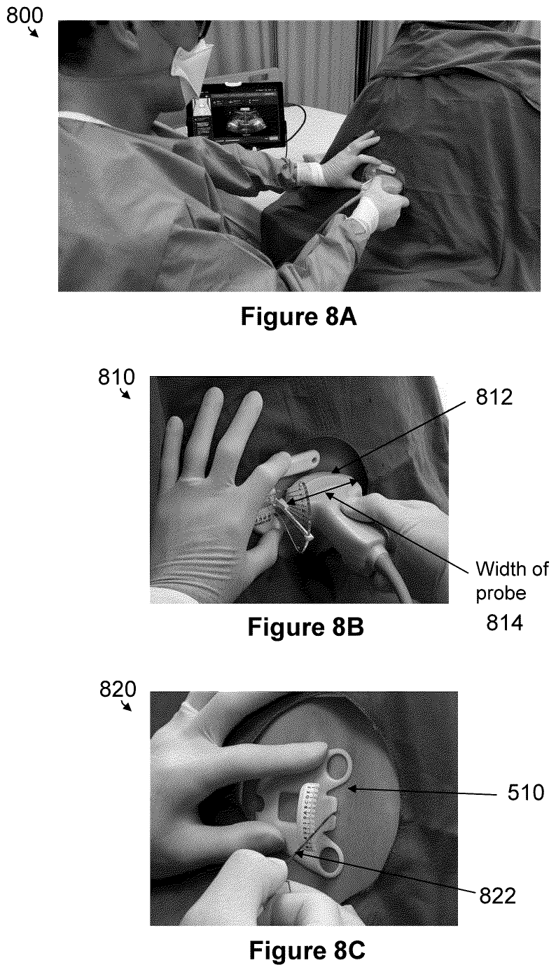

As shown in FIG. 6A, the angle scale base 610 together with the fan-shaped indicator plane 608 form a L-shape of the angle scale. Also shown in FIG. 6A is that the angle indicator 606 is operationally connected to the angle scale 604 via a pivot 612 at the angle scale base 610 which allows the angle indicator 606 to rotate about the pivot 612 for indicating a probe angle using the angle markings on the angle scale 604. More clearly shown in FIG. 5A, the angle indicator 606 is adapted to be aligned along a longitudinal mid-plane of a face of the imaging probe (i.e. the angle indicator is aligned along a plane which is parallel to the longitudinal axis of the face of the imaging probe and crosses a middle of the imaging probe) and is configured to move or rotate or tilt in tandem with the imaging probe as the imaging probe rotates to identify an insertion angle for needle insertion. This will be discussed in more detail in relation to FIGS. 8A and 8B below.

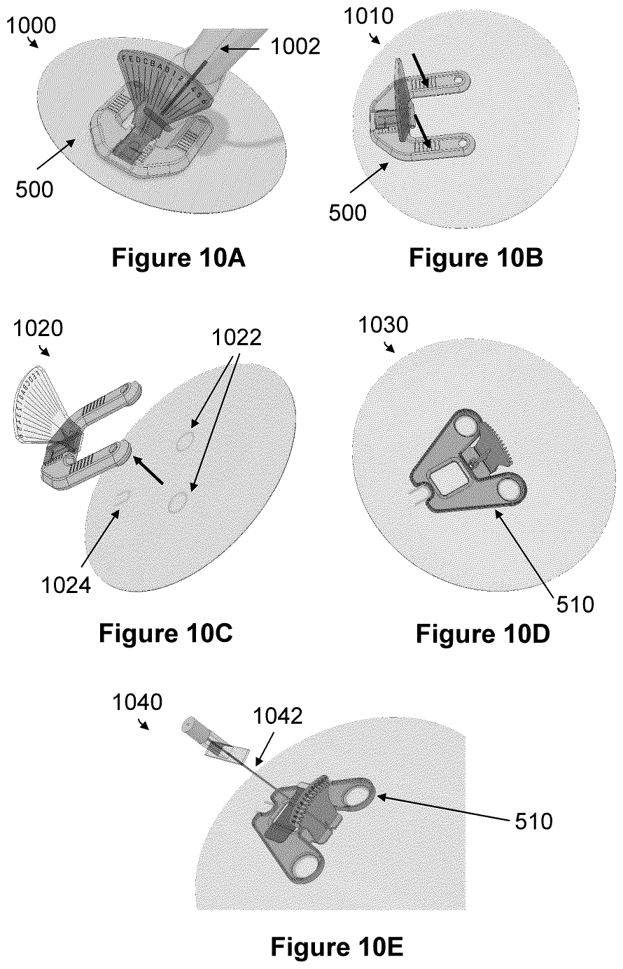

Further, the base 602 of the present embodiment comprises at least one protrusion 614 for generating at least one mark on a skin of the patient to indicate a position of the base with respect to the skin of the patient when the imaging probe is aligned at the insertion angle at the insertion site. This will be made apparent in the description of FIGS. 10A to 10E. Also shown in FIG. 6A is that the base 602 includes grooves 616 on a top surface of the prongs for the C-shaped base 602. The grooves 616 provide grips when pressing the marking device down on the skin of the patient for generating the at least one mark.

The different components of the marking device—the base 602, the angle scale 604, the angle indicator 606, are injection moulded in the present embodiment. In the present embodiment, the base 602 and the angle indicator 606 are made of acrylonitrile butadiene styrene (ABS) while the angle scale is made of polycarbonate.

As shown in FIG. 6B, the probe interface 620 (in the form of a connector) includes a connector base 622 and a groove 624. The connector base 622 includes an adhesive for attaching the probe interface 620 to a longitudinal mid-plane of the head of the imaging probe (see e.g. FIG. 5A). The groove 624 is adapted to be detachably fitted to the angle indicator 606 (see e.g. FIG. 5A where the angle indicator is fitted between the groove of the probe interface) for operationally connecting the imaging probe to the angle indicator. In this way, the angle indicator 606 moves in tandem with the imaging probe as the imaging probe is rotated to identify the optimal insertion angle on the angle scale 604. In the present embodiment, the probe interface may be made of acrylonitrile butadiene styrene (ABS).

The angle scale 604, the angle indicator 606, the base 602 and the probe interface 620 may be moulded in a same tool (or family tool) by injection moulding in the present embodiment.

FIGS. 7A and 7B show diagrams of the needle guide 510 of FIG. 5B in accordance with an embodiment, FIG. 7A shows a diagram of the needle guide 510 in a ready to use state and FIG. 7B shows a diagram of the needle guide 510 as formed (or in a moulded state).

As shown in FIG. 7A, the needle guide 510 comprises a guiding angle scale 702 having guide angle markings corresponding to the angle markings of the marking device for indicating the angle positions of the imaging probe, and an insertion slot 704 for aligning a needle to the insertion site. In the present embodiment, the guide angle markings therefore are of 5-degree increments, corresponding to that of the marking device. In the present embodiment, the guiding angle scale 702 also has a fan-shaped indicator plane, similar to that of the angle scale 604 of the marking device. Also shown in FIG. 7A is that the needle guide comprises a needle slot 706 at each of the guide angle markings. The needle slot 706 allows an alignment of the needle to a corresponding angle position, therefore ensuring that the needle is maintained at the insertion angle during needle insertion.

FIG. 7B shows a diagram of the needle guide 510 as formed (or in a moulded state). More clearly shown in FIG. 7B is that the needle guide 510 also comprises a hinge 708 as moulded adapted to allow the guiding angle scale 702 to hinge at a needle guide base so that the guiding angle scale is collapsible to be in plane with the needle guide base. This allows the needle guide to be manufactured and packaged in a compact, flat-packed manner which aids efficient transport and storage of the needle guide. The needle guide also has a bracket 710 and a corresponding protruding member 712 adapted to be locked in place by the bracket 710 (e.g. tongue and groove fitting/locking mechanism). To convert the moulded state of the needle guide as shown in FIG. 7B to the ready-to-use state as shown in FIG. 7A, the guiding angle scale 702 can be folded up at the hinge 708 and the corresponding protruding member 712 can be snapped in place in the bracket 710 for securing the guiding angle scale 702 in an upright position as shown in FIG. 7A.

Also shown in FIG. 7B is that the needle guide comprises at least one opening 714a, 714b, 714c at a needle guide base of the needle guide. In the present embodiment, the at least one opening 714a, 714b, 714c includes two circular openings 714a, 714b and a C-shaped opening 714c. These openings 714a, 714b, 714c are adapted to correspond to the at least one protrusion 614 of the base 602 of the marking device for aligning the needle guide to the insertion site using at least one mark made on the skin of the patient by the marking device. This is discussed in more detail in relation to FIGS. 10A to 10E below.

In the present embodiment, the needle guide 510 can be formed by injection moulding. In the present embodiment, the needle guide 510 is made of polypropylene. The needle guide 510 in the present embodiment includes a low-cost single piece moulding. The needle guide 510 can also be made sterile for ease of use during the needle insertion procedure as it is applied onto the injection site after the injection site is disinfected.

FIGS. 8A, 8B and 8C show illustrations of a method for marking the needle insertion site and the insertion angle using the needle guiding system of FIGS. 5A, 5B and 5C in accordance with an embodiment.

FIG. 8A shows an illustration 800 of determining a needle insertion site and an insertion angle using neuraxial ultrasonography. As shown in FIG. 8A, an ultrasound probe is first used to identify an optimal insertion site for the needle insertion. This can be performed using first a vertical scan to locate the interspinous space (e.g. the L3/L4 interspinous space) for the needle insertion, followed by a horizontal scan to identify the spinal midline and to align the spinal midline to a middle of the ultrasound machine monitor (and therefore a middle or centre of an ultrasound probe window) for identifying the insertion site. In the present embodiment, once the ultrasound probe is used to perform the horizontal scan, the marking device can be connected to the ultrasound probe using the connector as afore-described.

FIG. 8B shows an illustration 810 of marking the needle insertion site and recording the insertion angle using the marking device 500 of FIG. 5A. Once the marking device 500 is connected to the ultrasound probe 812, the clinician or the anesthesiologist performing the procedure optimizes a view of the selected interspace by tilting the ultrasound probe to locate the optimal angle for needle insertion. The angle of the ultrasound probe relative to the patient's skin at the optimized view of the selected interspinous space determines the required needle angulation during insertion. As described in relation to FIG. 6A, the angle indicator 606 of the marking device 500 moves in tandem with a rotation or tilting of the ultrasound probe 812 as the ultrasound probe 812 is aligned to identify the insertion angle. Therefore, once the insertion angle (or the optimal needle angulation) has been found using the ultrasound probe 812, this insertion angle can be identified by the angle indicator 606 on the angle scale 604. The identified insertion site can also be marked by pressing the marking device 500 onto the skin of the patient. In this case, the insertion site can be marked on the skin with respect to the markings generated by the at least one protrusion 614 of the base 602 of the marking device 500. The procedure of identifying the insertion site and insertion angle for the needle insertion is prior to needle insertion and therefore can be performed in a non-sterile environment. The marking device 500 in the present embodiment can therefore be non-sterile and reusable. Also shown in FIG. 8B is a width 814 of the probe, which is defined as a distance/length across the head/face of the imaging probe 812 and along a longitudinal axis of the head/face of the imaging probe 812.

FIG. 8C shows an illustration 820 of guiding a needle for needle insertion at the needle insertion site and at the insertion angle using the needle guide 510 of FIG. 5B. Using the marks formed on the skin of the patient, the needle guide 510 can be aligned using the corresponding openings 714a, 714b, 714c to the insertion site. The needle guide 510 and the marking device 500 of the needle guiding system are designed such that the insertion slot 704 is aligned at the insertion site (which corresponds to a “middle” of the probe window/face (i.e. an intersection of a longitudinal axis and a transverse axis of a probe window) of the ultrasound probe 812 when the ultrasound probe 812 is properly aligned at the insertion site—see the description in relation to FIGS. 2A, 2B and 2C for locating the insertion site) once the needle guide 510 is aligned to the marks formed on the skin of the patient using the corresponding openings 714a, 714b, 714c. As shown in FIG. 8C, once the needle guide 510 is aligned, a needle 822 can be guided using the guide scale markings to be inserted at the insertion angle (e.g. by using an appropriate needle slot 706 which corresponds to the insertion angle) and through the insertion slot 704 at the insertion site. In this way, precise position and angulation of the needle 822 for needle insertion can be achieved. As the needle guide 510 is to be placed on the skin of the patient for performing the needle insertion procedure, the needle guide 510 in the present embodiment is made to be sterile and for one-time use.

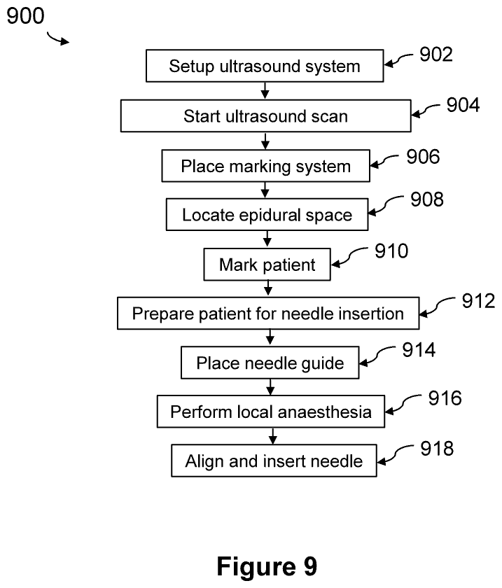

FIG. 9 is a flow chart showing steps of a method 900 for determining a needle insertion site using neuraxial ultrasonography and marking the needle insertion site using the needle guiding system of FIGS. 5A, 5B and 5C in accordance with an embodiment. As will be appreciated by the skilled person, the method 900 follows a similar general workflow as previously described in relation to FIGS. 1A to 1F, except for the method for marking the needle insertion site on the patient and guiding the needle to be inserted precisely and at an optimal angulation. This also means that the needle guiding system can be implemented clinically without disrupting a general workflow for a needle insertion procedure. Although FIG. 9 is described with respect to the needle guiding system of FIGS. 5A, 5B and 5C, it should be appreciated that the method 900 may also be applicable to other embodiments as described in the present disclosure.

In a step 902, an ultrasound machine or system is setup beside a patient, similar to FIG. 1A. In a similar manner to the workflow as described in FIGS. 1A to 1F, the patient can be seated with his/her back hunch in preparation for needle insertion.

In a step 904, ultrasound scans can be performed using the ultrasound system for locating a needle insertion site. At this stage, a clinician may apply ultrasound gel and scans the patient's lumbar area to look out for several spinal landmarks that will identify an optimal needle insertion spot and an insertion angle. Typically for epidural needle insertion, the landmarks are the L3/L4 interspinous space, a midline of the spine and an epidural space. In other embodiments where the needle guiding system is used for other applications, other suitable landmarks can be used.

In a step 906, the marking device of the needle guiding system is connected to the ultrasound probe. In the present embodiment of the method as being applied to the various embodiments of the marking device as described, the marking device is connected to the ultrasound probe once the vertical scan is performed to locate the desired interspinous space, and during the performance of the horizontal scan after a midline of the desired interspinous space has been located for identifying the optimal angle for needle insertion. In another embodiment of the method, the marking device can be connected to the ultrasound probe prior to performing the ultrasound scans. It should be noted that the user can connect the marking device to the ultrasound probe at any stage as appropriately as long as the marking device is fitted for recording the optimal insertion angle.

In a step 908, in the present embodiment, once the marking device is connected to the ultrasound probe, the epidural space is located. This can be performed using horizontal scans where the clinician optimizes a view of the selected interspace by tilting the ultrasound probe to locate the optimal angle for needle insertion. The angle of the ultrasound probe relative to the patient's skin at the optimized view of the selected interspinous space determines the required needle angulation during needle insertion. As described in relation to FIG. 6A, the angle indicator 606 of the marking device moves in tandem with a rotation or tilting of the ultrasound probe as the ultrasound probe is aligned to identify the insertion angle. Therefore, once the insertion angle (or the optimal needle angulation) has been found using the ultrasound probe, this insertion angle can be identified by the angle indicator 606 on the angle scale 604.

In a step 910, the identified insertion site is marked by pressing the marking device onto the skin of the patient. In this case, the insertion site can be marked on the skin with respect to the markings generated by the at least one protrusion 614 of the base 602 of the marking device.

In a step 912, the patient is prepared for needle insertion. This can be performed by disinfecting the insertion site using antiseptic solutions as shown in FIG. 1E. In the present case, the marks formed on the skin of the patient using the at least one protrusion 614 of the marking device do not fade or are not removed by disinfecting the insertion site.

In a step 914, a needle guide of the needle guiding system is placed. Using the marks formed on the skin of the patient, the needle guide can be aligned using the corresponding openings 714a, 714b, 714c to the insertion site.

In a step 916, local anaesthesia (LA) is performed at the insertion site. In the present embodiment, the clinician can perform LA when the needle guide is in a ready-to-use state (i.e. upright position) as shown in FIG. 7A where the LA can be injected precisely at the insertion site. It is noted that LA does not need to be injected at the insertion angle and so it can be inserted at an unblock side of the needle guide (e.g. at an opposite side to the guiding angle scale 702. In other embodiments (see e.g. the needle guide of FIG. 14A), the LA can be performed when the needle guide is in the moulded state (or flattened state) so that the guiding angle scale does not block the insertion of the LA needle.

In a step 918, once the needle guide is aligned, a needle can be aligned using the guide scale markings to be inserted at the insertion angle (e.g. by using an appropriate needle slot 706 which corresponds to the insertion angle) and through the insertion slot 704 at the insertion site. In this way, precise position and angulation of the needle for needle insertion can be achieved.

FIGS. 10A, 10B, 10C, 10D and 10E show diagrams illustrating a workflow for marking of the needle insertion site and the insertion angle using the needle guiding system of FIGS. 5A, 5B and 5C in accordance with an embodiment.

FIG. 10A shows a diagram 1000 illustrating alignment of the marking device 500 at the insertion site using the ultrasound probe 1002 and recording of the insertion angle. As described above, once the desired interspinous space is located by the ultrasound probe 1002 using vertical scans, the marking device 500 can be connected to the ultrasound probe 1002. The ultrasound probe 1002 is then tilted in the horizontal scans to determine an optimal angulation (i.e. an insertion angle) for needle insertion. Once the ultrasound probe 1002 is aligned to the insertion angle, the insertion angle can be recorded using the angle indicator of the marking device 500 by reading off the angle markings on the angle scale of the marking device 500.

After the insertion angle has been recorded, the marking device 500 is pressed against the skin of the patient for generating at least one mark on the skin of the patient using the at least one protrusion 614 of the base 602 of the marking device. This is shown in the diagram 1010 of FIG. 10B. The at least one mark formed on the skin of the patient can be used subsequently by the needle guide to locate the insertion site precisely.

FIG. 10C shows a diagram 1020 illustrating marks 1022, 1024 left on the skin of the patient using the marking device 500. In the present case, two circular marks 1022 and a C shape mark 1024 were used as the marks. The marking device 500 is then removed. As discussed earlier in relation to FIG. 9, the patient is then prepared for needle insertion, for example, by disinfecting the area around the insertion site. Once the patient is prepared, the needle guide 510 can be used as shown in FIG. 10D.

FIG. 10D shows a diagram 1030 illustrating alignment of the needle guide 510 of FIG. 5B at the insertion site using the marks 1022, 1024 left on the skin of the patient. The needle guide 510 is aligned to the insertion site using the corresponding openings 714a, 714b, 714c of the needle guide 510 and the at least one mark 1022, 1024 on the skin of the patient.

Once the needle guide is aligned, the needle 1042 for insertion can be aligned to the insertion angle using a needle slot 706 which corresponds to the identified insertion angle, and to the insertion site using the insertion slot 704. The needle 1042 can then be precisely inserted at the right angulation and at the correct insertion site identified using the ultrasound probe 1002. This is shown in the diagram 1040 of FIG. 10E.

Therefore, as described above, an embodiment of a needle guiding system (or a Position and Angle Marking System (PAMS)) that will address aforementioned limitations of neuraxial ultrasonography has been described. The needle guiding system is able to translate the information derived from ultrasonography into more precise needle insertion and angulation during the procedure, through its ability to precisely and rapidly demarcate the needle insertion point, as well as accurate measurement and maintenance of the required needle angulation. In addition, PAMS is compatible with a wide range of ultrasound probes (e.g. with different probe widths) and will therefore be applicable to a multitude of neuraxial procedures and settings worldwide. By increasing the accuracy of needle insertion and angulation, the safety and reliability of lumbar neuraxial procedures can be improved, thereby benefitting a large patient population and reducing healthcare-related costs.

The functions of the marking device and the needle guide of the needle guiding system are summarised below.

The functions of the marking device in the present embodiments include:
To make indentations on the patient's back to aid alignment of the needle guide;
To have a length adjustment mechanism to fit probes of varying sizes (lengths and/or widths);
To have a mechanical alignment mechanism for recording the insertion angle from the midline of the ultrasound probe;
To record the angle of the probe in relation to a skin of the patient (or effectively a base of the marking device).

The functions of the needle guide in the present embodiments include:
To position the needle at the insertion point/site using the marks made on the skin of the patient by the marking device;
To act as a sterile needle guide to maintain the optimal angulation during the procedure.

The key novel aspects of the needle guiding system or PAMS include:
Ability to record angle of the probe and maintain the needle at the same angle during insertion
Ability to make indentations or marks on a patient's skin for quick and clear marking that remain visible after skin sterilization procedure
Able to fit ultrasound probes of different sizes and/or shapes
Design of the needle guiding system or PAMS in the present embodiment include:
   Shapes and positions of indentation, including 2 circular and 1 U-shaped or C-shaped marks
   Adjustment mechanism to fit probes of varying window lengths or probe widths
   Design of the probe specific interface that is fitted to the specific probe to enable quick attachment and detachment of the marking device to the probe
   Design of the angle recording mechanism in the marking device (e.g. the angle scale and/or the angle indicator) with a pivot point to allow tilting movement of the probe
   Foldable design of the needle guide to (1) enable flat packaging, (2) allow clinicians to easily inject local anaesthesia (LA) at the insertion site as LA does not need to be at the same probe angle, and LA needle is typically shorter, hence the needle guide in the upright position (see e.g. FIG. 14B below) might block the insertion of LA needle.
   Design of the needle guide to have notches or needle slots to align a needle (e.g. a spinal needle or an epidural needle) at the correct angle (i.e. the identified insertion angle).

### Advantages of the Needle Guiding System (PAMS) Over Currently Available Technologies

As discussed above, PAMS addresses several limitations of neuraxial ultrasonography, including difficulties with precise demarcation of needle position and maintaining optimal needle angulation during the procedure. For example, PAMS replaces the conventional surgical marker used (see in relation to FIGS. 2A-2C and 3A-3B), for providing a precise and easy system to position and angulate needle for insertion.

### Functional and Usability Tests for First Prototype of the Needle Guiding System (PAMS)

With the first prototype, a functionality test was performed to verify that a first prototype of PAMS (i.e. the needle guiding system of FIGS. 5A-5C) met most if not all requirements as previously listed. Subsequently, a usability study involving 8 anesthesiologists and 3 simulated patients was conducted to obtain feedback and to identify areas for improvement of the first prototype. In this usability study, the anesthesiologists each performed two simulated neuraxial ultrasound procedures on healthy volunteers: one using standard clinical practice where marker pens were used to demarcate the needle insertion point, and the other using this first PAMS prototype to demarcate the needle insertion point and measure the required needle angulation.

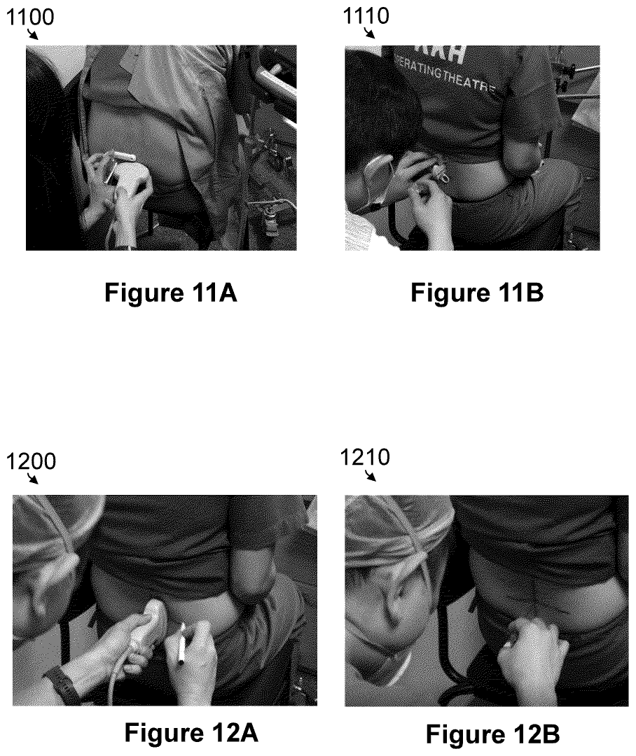

FIGS. 11A and 11B show illustrations of the usability study performed using the needle guiding system of FIGS. 5A, 5B and 5C. FIG. 11A shows an illustration 1100 of aligning the marking device 500 of FIG. 5A to an insertion site and recording an insertion angle and insertion site/point using the ultrasound probe, while FIG. 11B shows an illustration 1110 of a mock needle insertion using the needle guide 510 of FIG. 5B. In this case, the mock needle used was a blunt epidural needle.

FIGS. 12A and 12B shows illustrations of a usability study performed using the conventional method for determining of the needle insertion site with neuraxial ultrasonography and marking of the needle insertion site using a marker pen. FIG. 12A shows an illustration 1200 of marking the insertion site using the marker pen and FIG. 12B shows an illustration 1210 of a mock needle insertion with a blunt epidural needle by estimating the insertion angle.

The results of the usability study suggest that the first PAMS prototype was comparable to standard clinical practice in terms of overall usability and procedure time (PAMS: 141 seconds, marker pens: 142 seconds). PAMS scored better with demarcating the needle insertion point/site, determining the needle angulation, and overall satisfaction from the simulated patients (see Table 1). In addition, it was commented that PAMS integrated well into the current workflow of standard clinical practice for ultrasound-guided neuraxial procedures, and therefore minimal training and adjustment to the workflow were required for the anesthesiologists to use PAMS effectively.

TABLE 1
Overall usability test results for the first PAMS prototype
found to be superior to standard practice. All categories
were scored from 1 to 5, with 5 denoting the best experience
or satisfaction and 1 the worst

| | | Standard |
|---|---|---|
| Experience with | PAMS | practice |
| Experience with demarcating | 4.25 | 3.88 |
| needle insertion point | | |
| Satisfaction with demarcating | 4.25 | 3.62 |
| needle insertion point | | |
| Experience with determining | 4.13 | 3.50 |
| needle angulation | | |
| Satisfaction with determining | 4.13 | 3.50 |
| needle angulation | | |
| Experience of simulated patients | 4.70 | 4.30 |

Based on feedbacks from the usability study above, areas of improvement and refinement of the first PAMS prototype were identified. This includes: (1) simplifying a process of connecting the marking device to the ultrasound probe; (2) allowing easy and rapid detachment of the marking device from the ultrasound probe without affecting the angle measurements; and (3) improving the interface between the marking device and the ultrasound probe that does not impede movement of the ultrasound probe when obtaining the optimal image and needle angulation.

### Second and Subsequent PAMS Prototypes

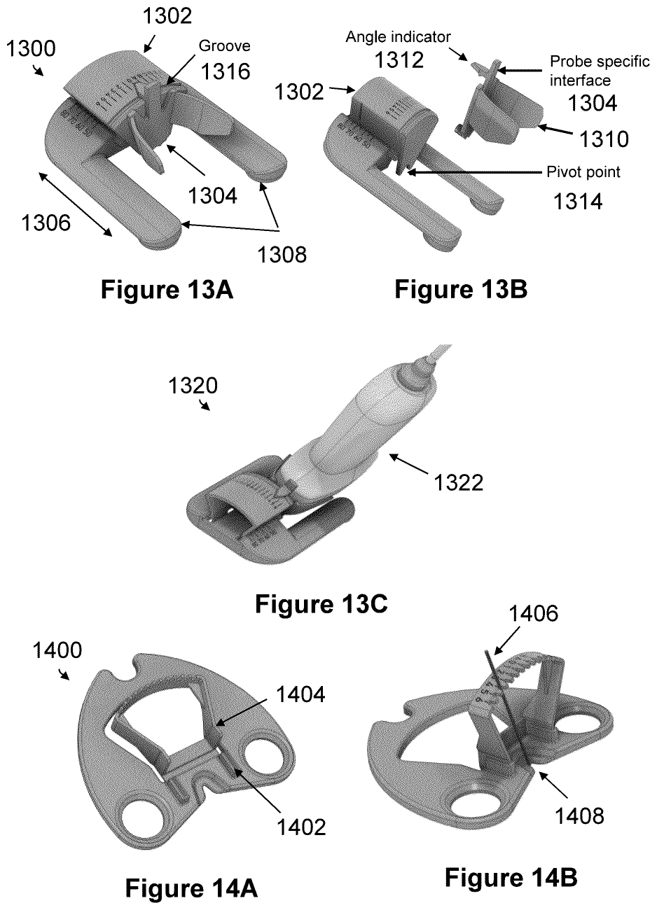

A second prototype was formed to improve on usability and ease-of use, especially pertaining to the probe interface and the design for connecting the probe to the marking device mounting design (see e.g. FIGS. 13A-13C). The needle guide has also been revised as shown in FIGS. 14A and 14B.

FIGS. 13A, 13B and 13C show diagrams of a marking device 1300 of a needle guiding system in accordance with a second embodiment. FIG. 13A shows a diagram of the marking device 1300 with a modified angle scale 1302 and a detachable probe interface 1304 in an assembled state, while FIG. 13B shows a diagram of the marking device 1300 with the modified angle scale 1302 and the detachable probe interface 1304 in a disassembled state. Compared to the first prototype as shown in relation to FIGS. 5A-5C, the angle scale 1302 has been configured to face the clinician while the clinician tilts the probe to identify the insertion angle. This improves a readability of the insertion angle and minimises a parallax error as compared to the marking device 500 of the first prototype. The angle scale 1302 of the second prototype is also slidable and adjustable, in a direction 1306 along the two prongs 1308 of the base, to cater for probes of different sizes (e.g. lengths or widths). Also shown in FIGS. 13A and 13B is that the probe interface 1304 of this second prototype includes (i) a probe holder 1310 which is configured to hold the ultrasound probe and stabilises the ultrasound probe and (ii) an angle indicator 1312 (see e.g. FIG. 13B). In this way, the ultrasound probe can be easily attached to the probe interface and also easily removed without affecting angle measurements. The probe interface 1304 is also adapted to be rotatable about a pivot point 1314 so that it moves in tandem with a tilting or rotation of the ultrasound probe (e.g. along a groove 1316 on the angle scale 1302) for identifying an insertion angle for needle insertion. The probe interface 1304 is also adapted to be detachable from the angle scale of the marking device 1300, as shown in FIG. 13B. FIG. 13C shows a diagram 1320 of the marking device 1300 with an ultrasound probe 1322 attached.

FIGS. 14A and 14B show diagrams of a needle guide of the needle guiding system in accordance with the second embodiment. FIG. 14A shows a diagram of the needle guide 1400 which is folded flat for packaging and storage. FIG. 14B shows a diagram of the needle guide 1400 with its guiding angle scale pushed up and fitted with a click in a ready-to-use state. As shown in FIG. 14A, means for securing the guiding angle scale to the needle guide base includes two brackets/grooves 1402 with corresponding tongues/fitting members 1404. FIG. 14B also shows that a needle 1406 being fitted into the needle slot 4 and inserted through the insertion slot 1408 of the needle guide 1400. The needle guide 1400 of this second embodiment is also in the form of an arch and is adapted to allow the insertion site to be visible from both sides of the arch. A skilled person will appreciate that other forms of the needle guide can also be used as long as it is able to guide the needle to be inserted at the insertion angle and to mark the insertion site for needle insertion.

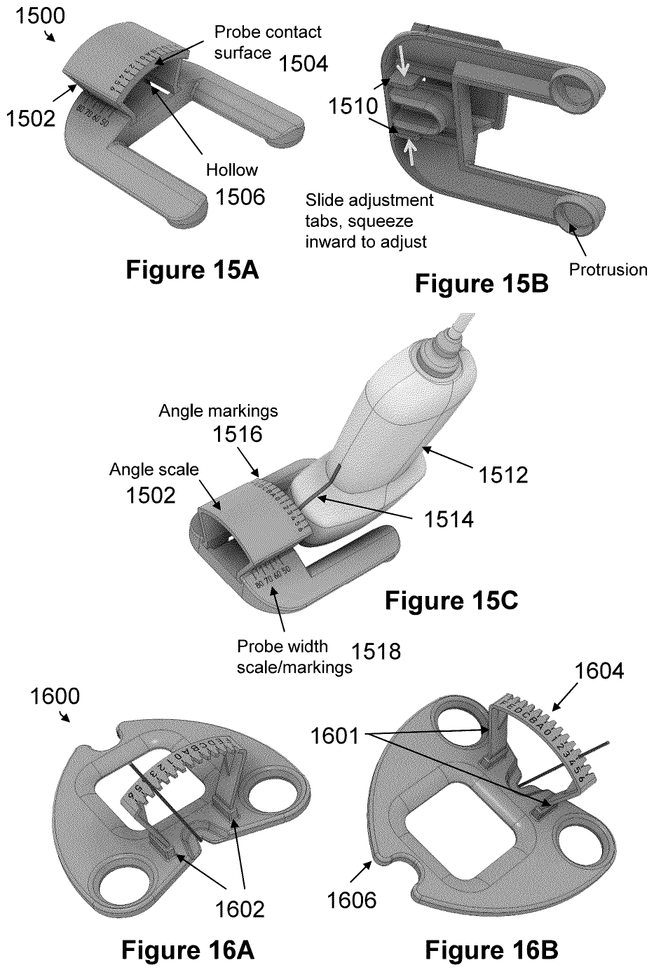

FIGS. 15A, 15B and 15C show diagrams of a marking device 1500 of a needle guiding system in accordance with a third embodiment. FIG. 15A shows a diagram of a perspective view of the marking device 1500 with a modified angle scale 1502. As shown in FIG. 15A, angle markings of the angle scale 1502 for this third embodiment are aligned to an inner edge of the angle scale at a probe contact surface 1504. Main differences between the angel scale of this third embodiment and that of the second embodiment are that: (i) absence of a groove 1316 at the angle markings of the angle scale 1502 in this case and (ii) a side of the angle scale (at the side of the probe contact surface) is hollow 1506 as compared to a solid side of the angle scale 1302 with a pivot point 1314 of the second embodiment as shown in FIG. 13B). FIG. 15B shows a diagram of a bottom view of the marking device 1500 to illustrate an adjustability of the angle scale 1502 for imaging probes of different sizes (e.g. different probe widths or lengths of the probe window). In the present embodiment, a base of the angle scale includes two slide adjustment tabs 1510 which can be squeezed inward for sliding the angle scale 1502 to adjust the angle scale 1502 to a suitable position for a probe width. FIG. 15C shows a diagram of the marking device 1500 aligned beside an imaging probe 1512 for direct reading of an insertion angle. In this case, an angle indicator 1514 of the marking device can be attached/formed at a side of a probe window of the imaging probe 1512 on a side of the angle scale 1502 as shown in FIG. 15C. Angle markings 1516 and probe width scale/markings 1518 are also shown in FIG. 15C. In the present embodiment, the probe 1512 can be in contact with the angle scale 1502 while being tilted/adjusted to identify an insertion angle for direct reading off the angle scale 1502.

FIGS. 16A and 16B show diagrams of a needle guide 1600 of the needle guiding system in accordance with the third embodiment to illustrate that a needle for needle insertion is visible at both sides of an arch of the needle guide 1600. In the present embodiment, similar to the needle guide 1400 of the second embodiment as shown in FIG. 14B, a guiding angle scale of the needle guide 1400 is in the form of an arch which allows placement of the needle at the insertion slot of the needle guide 1600 to be visible from both sides of the arch. This improves a visibility of the needle during needle insertion. Also shown in FIGS. 16A and 16B are in place of a live-hinge as exemplified by the needle guide of the second embodiment, the needle guide 1600 of this third embodiment shows that the guiding angle scale includes two legs 1601 which can be directly fitted into the slots 1602 formed at the base of the needle guide 1600 for assembling this needle guide 1600. It should be appreciated therefore that the needle guide 1600 of this third embodiment may be fabricated in a two-component form, i.e. a guiding angle guide 1604 and a needle guide base 1606, where the guiding angle guide 1604 can be subsequently connected to the needle guide base 1606 in the ready-to-use state using the slots 1602.

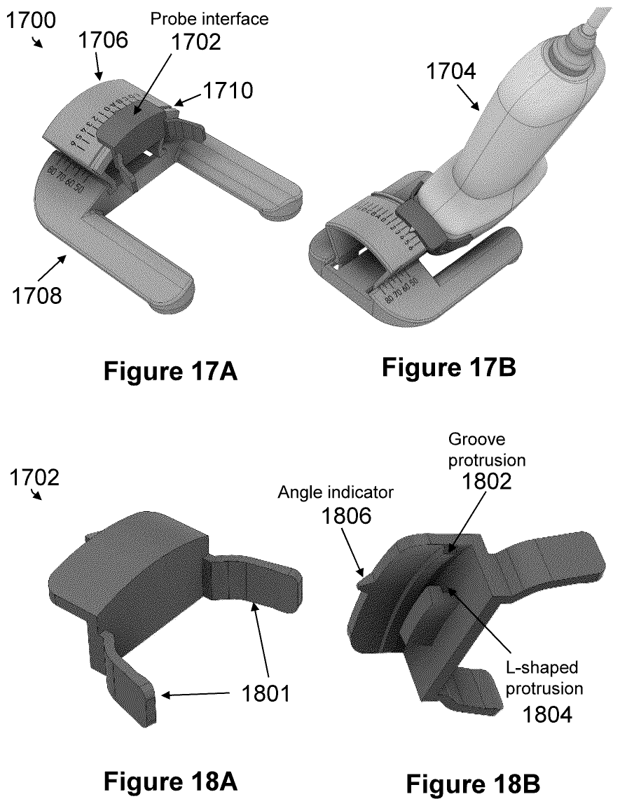

FIGS. 17A and 17B show diagrams of a marking device of a needle guiding system in accordance with a fourth embodiment. FIG. 17A shows a diagram of a perspective view of the marking device 1700 with a detachable probe interface 1702 in an assembled state, and FIG. 17B shows a diagram of the marking device 1700 with an imaging probe 1704 attached using the detachable probe interface. The angle scale 1706 and the base 1708 of the marking device 1700 of this fourth embodiment are similar to those of the third embodiment as shown in relation to FIGS. 15A and 15B, except that in this case, the groove 1710 at the angle markings of the angle scale 1706 is provided (similar to that as shown in FIG. 13A) and that the angle markings are not formed at the edge of the angle scale 1706. In this present case, a L-shaped groove is also formed at the probe contact side of the angle scale at an underside of the angle scale for complementing use of the probe interface. Another difference in this fourth embodiment relates to a shape of the probe interface 1702 where it is designed to translate along the groove 1710 formed on the angle scale 1706. This is in contrast to the probe interface 1304 of the second embodiment as shown in FIG. 13B which is designed to rotate about the pivot point 1314 of the angle scale 1302 of the second embodiment. The mechanism for having the probe interface 1702 to move in tandem with the tilting of the probe is therefore different for the second and fourth embodiment. A skilled person will understand that a shape of the probe interface can be customised to a probe shape as required.

FIGS. 18A and 18B shows diagrams of the detachable probe interface 1702 of the marking device 1700 of FIG. 17A in accordance with the fourth embodiment, where FIG. 18A shows a diagram illustrating a probe side of the detachable probe interface 1702 for attaching an imaging probe and FIG. 18B shows a diagram illustrating a marking device side of the detachable probe interface 1702 for attaching the probe interface to the marking device 1700. As shown in FIG. 18A, the detachable probe interface 1702 includes two resilient probe interface legs 1801 serve to hold or connect the imaging probe 1704 in place. As shown in FIG. 18B, the marking device side of the detachable probe interface 1702 has a groove protrusion 1802 which fits onto the groove 1710 of the angle scale 1706 of FIG. 17A, and a L-shape protrusion 1804 which fits to the L-shaped groove at the probe contact side of the angle scale 1706. These features enable the detachable probe interface 1702 of this fourth embodiment to be slidable with respect to the angle markings of the angle scale 1706 for indicating an insertion angle when the probe is aligned at the insertion angle for needle insertion. Also shown in FIG. 18B is an angle indictor 1806 of the detachable probe interface 1702. The angle indicator 1806 serves to indicate a corresponding angle marking on the angle scale to identify a tilting angle of the imaging probe 1704.

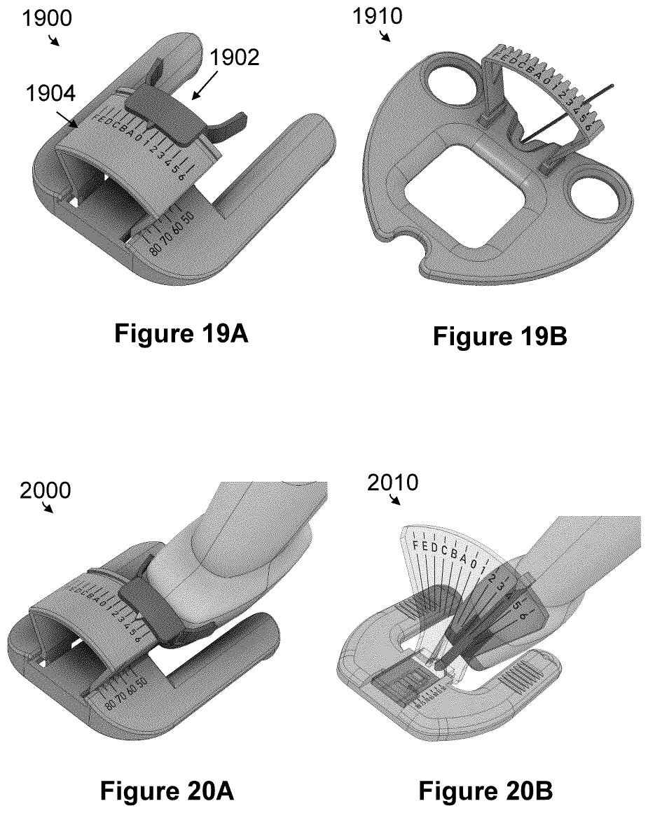

FIGS. 19A and 19B show diagrams of the needle guiding system in accordance with the fourth embodiment, where FIG. 19A shows the marking device 1900 of the needle guiding system and FIG. 19B shows a needle guide 1910 of the needle guiding system. The marking device 1900 of FIG. 19A as shown is in an assembled state which includes the detachable probe interface 1902 being movably connected to the angle scale 1904, which is similar to that shown in FIG. 17A. The description in relation to FIGS. 17A-17B and 18A-18B applies in this case. FIG. 19B shows the needle guide 1910 of the needle guiding system of the fourth embodiment. This is similar to the needle guide 1600 of the third embodiment as shown in relation to FIGS. 16A and 16B. It should be noted that the marking device 1900 and the needle guide 1910 of a same embodiment can be formed to have similar appearance or designs (e.g. that the arch of the angle scale of the marking device and the arch of the guiding angle scale of the needle guide can be formed using similar colour and having similar dimensions etc.) which may aid users of the needle guiding system in identifying them.

FIGS. 20A and 20B shows diagrams of two different embodiments of the marking device (i.e. the first embodiment and the fourth embodiment) for comparison. For example, it is shown in relation to FIGS. 20A and 20B, the marking device 2000 of the fourth embodiment includes angle markings which are easier to read as they are facing the clinician (e.g. in plane of the probe window of the imaging probe) as compared to the marking device 2010 of the first embodiment, and there is no parallax error due to transparent surfaces of the angle scale.

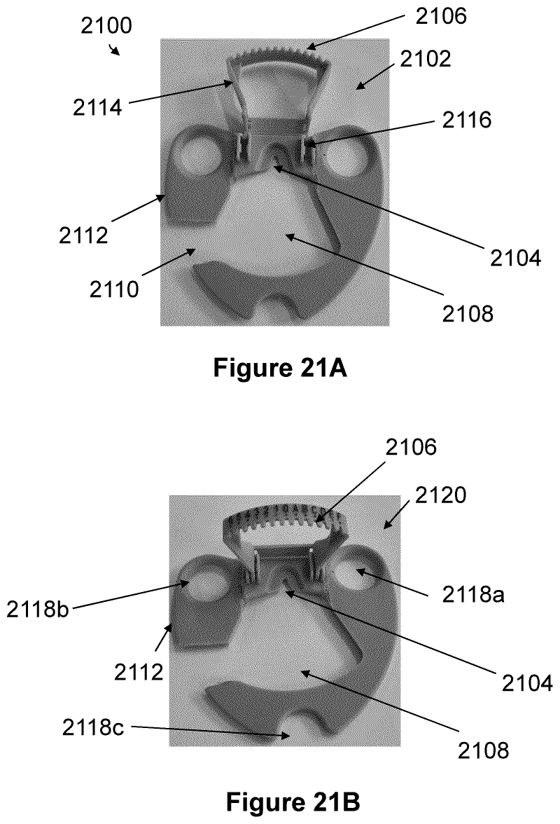

FIGS. 21A and 21B show photographs of a needle guide in accordance with a fourth embodiment, where FIG. 21A shows a photograph of the needle guide 2100 which is folded flat 2102 for packaging and storage, and FIG. 21B shows a photograph of the needle guide 2100 with its guiding angle scale pushed up and fitted with a click in a ready-to-use state 2120.

This fourth embodiment of the needle guide 2100 is similar to the second embodiment as shown in relation to FIGS. 14A and 14B, except that: (i) the insertion slot 2104 and the needle slots 2106 are faced inward toward a cavity 2108 of the needle guide 2100 and (ii) there is a gap/slot 2110 at the needle guide base 2112. The gap/slot 2110 allows the needle guide 2100 to be removed/dislodged once the needle has been injected. The present embodiment provides a better visual of the insertion slot 2104 together with the needle slots 2106 during insertion of the needle as compared to earlier embodiments of the needle guide. Similar to the needle guide as shown in relation to FIG. 14A, means for securing the guiding angle scale 2114 to the needle guide base 2112 includes two brackets/grooves 2116 with corresponding tongues/fitting members. The needle guide of this fourth embodiment is also in the form of an arch and is adapted to allow the insertion site to be visible from both sides of the arch. The other functions of the needle guide 2100, for example the openings 2118a, 2118b, 2118c adapted to correspond to the at least one protrusion of the base of a marking device for aligning the needle guide to the insertion site using at least one mark made on the skin of the patient by the marking device, are similar to that described in relation to the second embodiment of the needle guide and are not further described here for succinctness.

The embodiments of the needle guiding system as described above includes mostly plastic parts that require precision. Injection moulding may be one of the suitable methods for use in this case, given that injection moulding is capable of mass production of products with complicated shapes with good precision. The moulded product (e.g. the base, the angle scale and the probe interface of the marking device and/or the needle guide) is manufactured by injecting plastic materials molten by heat into a mould, and then cooling and solidifying them. The injection moulding machine includes an injection unit, the mould and a fixture that clamps the mould and releases the moulded plastic product when cooled. Injection moulding provides several advantages such as fast production, high precision and repeatability, supports complex part design and flexibility to choose a wide variety of materials. However, the disadvantage of injection moulding is its initial high setup cost to manufacture the moulding tool. Once the injection moulding process is set up, each moulding tool could support 5,000 to 10,000 production units, and this would represent cost-saving over time.

Besides the manufacturing method, considerations are also need for the selection of a right material (considering chemical, physical, toxicity, biocompatibility properties) and a right sterilization method (e.g. sterilization evaluation according to ISO 10993-1:2018) for the needle guide. Besides costs of materials and sterilization, other related costs of fabrication include pad printing for the labels on PAMS, packaging and logistics. The envisioned needle guiding system as formed includes a plastic consumable that is used during any procedure that uses an imaging technique (e.g. ultrasound) to determine a needle insertion position and an angle orientation. To be suitable in a clinical setting, the non-sterile marking device and the sterile needle guide can be packaged individually in separate bags (e.g. polybag) which can be extracted easily for use during a needle insertion procedure.

Although embodiments of the needle guiding system as described above show that a marking device and a needle guide of the needle guiding system can be used together to achieve the various advantages as described, it should be appreciated that the marking device and/or the needle guide can also be used independently. For example, a marking device can be adapted to use to locate an insertion site and an insertion angle for needle insertion, followed by detaching of the probe and inserting of the needle as guided by the angle scale of the marking device. In another example, identification of an insertion site and/or an insertion angle can be achieved by other means, and a needle guide can be used independently for guiding a needle for the needle insertion. Further, although the above embodiments of the needle guiding system are described in relation to neuraxial procedures, it should be appreciated that application of the needle guiding system is not to be limited as such, and can include other procedures which may involve needle insertion.

It should be appreciated that, in an embodiment, a marking device of a needle guiding system can include an angle scale which is not detachable and/or slidable with respect to a base of the marking device. In this case, the angle scale is integrated with the base of the marking device to form a single-piece, and so each marking device may be made specific to a specific probe size. In a similar vein, it should also be appreciated that in an embodiment, the guiding needle scale is integrated with the needle guide base so that the needle guide is formed as a single piece and in a ready-to-use state. In this case, for example, the needle guide may form an extendable part of the marking device on an opposite side of the angle scale where it can be extended away to make way for the probe while the probe is in use to identify the insertion site and the insertion angle, and where it can be retracted in place so that an insertion slot of the needle guide marks the insertion site and the guiding angle scale can be used to guide the needle at the insertion angle for needle insertion.

It should also be appreciated that although the angle indicator as shown in the various aforementioned embodiments are aligned along the longitudinal mid-plane of the face of the imaging probe, in other embodiments, the angle indicator can be aligned at different positions with respect to the imaging probe as long as an insertion angle for needle insertion can be marked/recorded appropriately on the angle scale and be translated accurately/correctly to a corresponding guide angle markings of the needle guide for use in the needle insertion.

In other embodiments, the needle guiding system comprising the marking device and the needle guide may be adapted to measure and/or record two (or three) angles (i.e. angles in two or three dimensions, e.g. with respect to the Cartesian coordinates) in relation to the insertion angle of the needle. These embodiments may be helpful in applications where the needle is to be inserted at an angle e.g. away from a longitudinal plane of the spine or for paramedian insertion of the needle at a thoracic region of a patient. The embodiments of the invention are therefore also not intended to be limited to lumbar neuraxial procedures, and may be applicable to any applications which require precise needle insertion and/or guidance.

In the present embodiments as described in relation to FIGS. 4 to 21B, a pitch of the imaging probe is measured (e.g. an angle measured with respect to the longitudinal axis of the spine, i.e. z-axis). In embodiments where two angles in relation to needle insertion are measured, e.g. a yaw angle of the imaging probe (e.g. an angle measured with respect to the transverse axis of the spine, where the imaging probe is being tilted left or right with respect to a back of the patient) is also measured in addition to the pitch angle. In other words, an insertion angle for needle insertion in these embodiments are measured in two directions (or orthogonal directions). In these cases, a ball joint capable of rotating three-dimensionally can be connected to a base of the marking device. The ball joint may have angle markings for measuring the pitch angle and the yaw angle. Otherwise, the ball joint may include indicators that can indicate the pitch angle and the yaw angle on separate angle scales. Further, the needle guide in these embodiments may further include a secondary guiding angle scale adapted to indicate the yaw angle, in addition to the guiding angle scale as shown in the embodiments above which is adapted to indicate the pitch angle. For example, the guiding angle scale can be tilted systematically with respect to the secondary guiding angle using a hinge joint and can be adapted to be locked in place for aligning the needle at the appropriate pitch angle and yaw angle for needle insertion at the insertion site. In a similar vein, the skilled person would appreciate that the marking device and/or the needle guide can be configured or adapted to measure and guide a needle for insertion three-dimensionally (i.e. with respect to three angles).

Other alternative embodiments include: (1) a patient being a human or an animal; (2) an angle scale of a marking device being integrated with a base of the marking device; (3) no grooves on a base of the marking device; (4) a base of the marking device formed in other shapes (e.g. H-shape or U-shape) other than the C-shape as shown in the embodiments; (5) only one protrusion for providing one mark, in this case the mark can be designed to be asymmetrical so that an orientation of the base can still be identified and aligned using a corresponding opening in the needle guide, further a different number of protrusions (e.g. 2 or 4 etc.) and/or different protrusion shapes for each of the protrusions can also be used; (6) other imaging probes other than an ultrasound probe, as long as the imaging probe or technique allows for the identification of the injection site/point and the injection angle for needle insertion; (7) other suitable materials (e.g. bio-compatible material or metal) for making the needle guiding system other than plastic and/or those as described in the aforementioned tables; (8) other form of manufacturing method (e.g. thermoforming, compression moulding, blow moulding or casting method), other than injection moulding, for forming one or more of the marking device, the needle guide and the probe interface of the needle guiding system; (9) the guiding angle scale being adapted to hinge at a needle guide base and/or detachable from a needle guide base; (10) other securing means or locking mechanisms for holding the needle guide or the guiding needle scale in an upright state or ready-to-use state (e.g. lock and click, locking hinge etc.); (11) an angle scale of a marking device having one or more adjustment tabs for adjusting a position of the angle scale with respect to the base of the marking device to cater for different probe sizes; (12) graphics on the marking device and/or the needle guide (e.g. angle markings, probe width markings and/or guide angle markings) being screen printed or tampo printed or formed by other imprinting means; (13) angle positions of the angle markings of the marking device being regularly spaced at an angular displacement other than 5 degrees, for example at an angular displacement of 2 degrees, 4 degrees or 6 degrees or any other suitable angular displacement; (14) a needle guide having no needle slot at each of the guide angle markings; (15) a marking device and a needle guide being used independently and therefore the one or more protrusion at a base of the marking device and the one or more corresponding opening of the needle guide are optional; and (16) probe interface which is specific to a shape and/or size of an imaging probe used.

Although only certain embodiments of the present invention have been described in detail, many variations are possible in accordance with the appended claims. For example, features described in relation to one embodiment may be incorporated into one or more other embodiments and vice versa.

1. A needle guiding system comprising a marking device, wherein the marking device comprises:
a base;
an angle scale having angle markings indicating angle positions of an imaging probe with respect to a plane of the base; and
an angle indicator adapted to indicate a probe angle on the angle scale, wherein the angle indicator is configured to move in tandem with the imaging probe to identify an insertion angle for needle insertion when the imaging probe is aligned at the insertion angle at an insertion site.

2. The system of claim 1, further comprising a probe interface adapted to operationally connect the imaging probe to the angle indicator.

3. The system of claim 2, wherein the probe interface is adapted to be detachable from the angle scale.

4. The system of claim 2, wherein the probe interface includes a probe holder configured to hold the imaging probe and comprises the angle indicator.

5. The system of claim 1, wherein the angle scale is adapted to be movable with respect to the base so that an axis passing through a middle of the imaging probe is aligned to the insertion site for different widths of the imaging probe.

6. The system of claim 5, wherein the base further comprises width markings corresponding to respective positions of the movable angle scale for indicating a width of the imaging probe.

7. The system of claim 1, further comprising a needle guide, wherein the needle guide comprises:
a guiding angle scale having guide angle markings corresponding to the angle markings of the marking device for indicating the angle positions of the imaging probe; and
an insertion slot for aligning a needle to the insertion site.

8. The system of claim 7, wherein the needle guide further comprises needle slots at each of the guide angle markings for aligning the needle to a corresponding angle position.

9. The system of claim 7, wherein the base comprises at least one protrusion for generating at least one mark on a skin of the patient to indicate a position of the base with respect to the skin of the patient when the imaging probe is aligned at the insertion angle at the insertion site.

10. The system of claim 9, wherein the needle guide further comprises at least one opening corresponding to the at least one protrusion of the base for aligning the needle guide to the insertion site using the at least one mark on the skin of the patient.

11. The system of claim 7, wherein the guiding angle scale is adapted to hinge at a needle guide base so that the guiding angle scale is collapsible to be in plane with the needle guide base.

12.-14. (canceled)

15. The system of claim 1, wherein the angle positions are regularly spaced at an angular displacement of 5 degrees.

16. (canceled)

17. The system of claim 2, wherein the probe interface is adapted to be customisable to fit imaging probes of various shapes and sizes.

18. The system of claim 1, wherein the angle indicator is adapted to be aligned along a longitudinal mid-plane of a face of the imaging probe.

19. A needle guide comprising:
a guiding angle scale having guide angle markings adapted to indicate angle positions of an imaging probe for identifying an insertion angle for needle insertion; and
an insertion slot for aligning a needle to an insertion site for the needle insertion.

20. The needle guide of claim 19, further comprising needle slots at each of the guide angle markings for aligning the needle to a corresponding angle position.

21. The needle guide of claim 19, wherein the guiding angle scale is adapted to hinge at a needle guide base so that the guiding angle scale is collapsible to be in plane with the needle guide base.

22. The needle guide of claim 19, wherein the guiding angle scale is detachable from a needle guide base.

23. The needle guide of claim 19, wherein the guiding angle scale is in the form of an arch and is adapted to allow the insertion site to be visible from both sides of the arch.

24. A method of using a needle guiding system for guiding a needle for needle insertion of a patient, the needle guiding system comprising a marking device and a needle guide, wherein the marking device comprises a base, an angle scale having angle markings indicating angle positions of an imaging probe with respect to a plane of the base and an angle indicator adapted to indicate a probe angle on the angle scale, wherein the angle indicator is configured to move in tandem with the imaging probe, and wherein the needle guide comprises a guiding angle scale having guide angle markings corresponding to the angle markings of the marking device for indicating the angle positions of the imaging probe and an insertion slot for aligning a needle to an insertion site, wherein the base of the marking device comprises at least one protrusion and the needle guide further comprises at least one opening corresponding to the at least one protrusion of the base, the method comprising:
aligning the imaging probe to the insertion site and at an insertion angle;
using the angle indicator of the marking device to record the insertion angle associated with the angle markings of the angle scale;
generating at least one mark on a skin of the patient using the at least one protrusion of the base of the marking device for marking the insertion site with respect to the base of the marking device;
replacing the marking device with the needle guide;
aligning the needle guide to the insertion site using the at least one opening of the needle guide and the at least one mark on the skin of the patient; and
placing the needle in the insertion slot of the needle guide and at a guide angle marking corresponding to the insertion angle to guide the needle for needle insertion of the patient.
